(19) United States
(12) Patent Application Publication
(10) Pub. No.: US 2007/0190098 A1
(43) Pub. Date: Aug. 16, 2007

(54) Rodenticide

(75) Inventor: Joseph H. Henshaw, Nicosia (CY)

Correspondence Address: MORGAN LEWIS & BOCKIUS LLP, 1111 PENNSYLVANIA AVENUE NW, WASHINGTON, DC, 20004, US

(73) Assignee: Delmar Products Ltd.

(21) Appl. No.: 11/593,140

(22) Filed: Nov. 6, 2006

Related U.S. Application Data
(63) Continuation of application No. 10/456,734, filed on Jun. 9, 2003, now abandoned.
Application No. 10/456,734 is a continuation of application No. 09/393,261, filed on Sep. 10, 1999, now abandoned.
Application No. 09/393,261 is a continuation of application No. PCT/GB97/00800, filed on Mar. 21, 1997.

(30) Foreign Application Priority Data
Mar. 27, 1996 (GB) GB 9606483.7

(51) Int. Cl. A01N 65/00; A61K 36/899

(52) U.S. Cl. 424/410; 424/84; 424/750

(57) ABSTRACT

The invention provides a rodenticide comprising cellulosic material which is non-toxic to humans but which causes rodents to excrete body fat and/or adipose tissue. Suitable material is obtainable from the core of the cob of maize hybrid DK 446, a hybrid characterised by normally growing to a height of 2.7 to 3.3 metres (9 to 11 feet) and by normally having a single giant ear of corn. The cellulosic material is mixed with a bait attractant such as sugar beet or unrefined molasses and formed into pellets. The rodenticide is non-toxic when fed at controlled dosage levels (e.g. up to 15 g/kg per day) but toxic when the rats are allowed free access. Maize hybrids DK 401, DK 442, DK 512, DK 560, DK 588, DK 591, DK 604, DK 628, DK 634 and DK 512wx may also be used instead of hybrid DK 446.

The present invention relates to rodenticides.

It has unexpectedly been discovered that the cellulosic material obtainable from the core of the cob of a certain hybrid of maize (Zea Mays, known as corn in the USA) is toxic to rodents but not to humans.

This hybrid is known as DK 446 and is obtainable from Dekalb Plant Genetics (3100 Sycamore Rd, DeKalb, Ill. 60115 USA). It normally grows to a height of 2.7 to 3.3 metres (9 to 11 feet) and normally has a single giant ear of corn. It is commonly grown for use as cattle feed.

Accordingly in one aspect the invention provides a rodenticide comprising rodenticidal material obtainable from the core of the cob of maize hybrid DK 446.

The cellulosic material obtained from the core of the cob of the above hybrid has been analysed and has been found to consist of essentially pure α cellulose, which is the naturally occurring form of cellulose. α cellulose is generally recognised as a safe food additive with essentially no toxicity to humans and domestic animals.

Without wishing to be bound by theory, it is believed that rats and mice lose body fat and adipose tissue by excretion in the faeces and urine after eating the above cellulosic material, and eventually starve to death.

Accordingly, in another aspect the invention provides a rodenticide comprising cellulosic material which is non-toxic to humans but which causes rodents to excrete body fat and/or adipose tissue.

It is believed that the different effects on rodents and humans may be due to their different digestive systems, but the precise mode of action of the above rodenticidal material is currently unknown.

Other hybrids similar to the above DK 446 hybrid (especially maize hybrids characterised by normally growing to a height of 2.7 to 3.3 metres (9 to 11 feet) and by normally having a single giant ear of corn) are envisaged to be useful sources of identical or similar cellulosic rodenticidal material which is also usable in rodenticides in accordance with the invention. Furthermore hybrids DK 401, DK 442, DK 512, DK 560, DK 588, DK 591, DK 604, DK 628. DK 634 and DK 512wx, all obtainable from Dekalb Plant Genetics, are envisaged to be useful sources of identical or similar rodenticidal material.

Preferably the rodenticide of the present invention incorporates or is associated with a bait attractant such as a sweet material, eg ground sugar beets or unrefined (“black strap”) molasses, although any conventional bait attractant can be employed.

The cellulosic material can be obtained by tumbling the cob of a hybrid of the above DK 446 hybrid (although it is envisaged that similar hybrids eg, hybrids DK 401, DK 442, DK 512, DK 560, DK 588, DK 591, DK 604, DK 628, DK 634 and DK 512wx are also useful) to remove the reddish-brown outer layer, separating the outer layer material from the white hard core material of the cobs, and crushing the white core material, eg to the consistency of sawdust. The resulting powdered white hard core material is then preferably mixed with the bait attractant (eg unrefined molasses or ground dehydrated sugar beets). Preferably the bait attractant comprises 0.3% to 5%, more preferably 1% by weight of the mixture. The mixture is then preferably extruded eg at 30 to 1,000 MPa, preferably 324 MPa (47,000 psi) and the extrudate (which is eg of circular cross-section, with a diameter of eg 4 to 12 mm, preferably 10 mm diameter) can then be cut into pellets with a length of eg 10 to 30 mm, preferably 25 mm.

The above method of preparation can be varied however. For example the bait attractant could be coated on the extrudate or pellets instead of or in addition to being incorporated in the mixture before extrusion. Accordingly the invention also encompasses any rodenticide comprising cellulosic material (preferably the white hard core material) obtainable from the cob of any of the above hybrids (preferably DK 446, but possibly one or more of hybrids DK 401, DK 442, DK 512, DK 560, DK 588, DK 591, DK 604, DK 628, DK 634 and DK 512wx as well as any maize hybrids characterised by normally growing to a height of 2.7 to 3.3 metres (9 to 11 feet) and by normally having a single giant ear of corn).

In another aspect the invention provides a method of alleviating rodent infestation (particularly infestation by rats and mice), the method comprising depositing in the region of infestation a rodenticide comprising cellulosic material (preferably the white hard core material) obtainable from the cob of any of the above hybrids (preferably DK 446, but possibly one or more of hybrids DK 401, DK 442, DK 512, DK 560, DK 588, DK 591, DK 604, DK 628, DK 634 and DK 512wx as well as any maize hybrid characterised by normally growing to a height of 2.7 to 3.3 metres (9 to 11 feet) and by normally having a single giant ear of corn).

The invention also provides a method of making a rodenticide comprising the step of bringing into association a) a bait attractant and b) cellulosic material (preferably the white hard core material) obtainable from the cob of any of the above hybrids (preferably DK 446, but possibly one or more of hybrids DK 401, DK 442, DK 512, DK 560, DK 588, DK 591, DK 604, DK 628, DK 634 and DK 512wx as well as any maize hybrids characterised by normally growing to a height of 2.7 to 3.3 metres (9 to 11 feet) and by normally having a single giant ear of corn).

The non-toxic nature of the rodenticide of the invention has in fact been proved in the following study involving the controlled administration of predetermined amounts of the rodenticide to rats:

Toxicity Study

TEST ANIMALS: Sprague Dawley derived Rattus norvegicus

NUMBER AND SEX (in each group): 5 Male & 5 Female (females nulliparous and non-pregnant)

NUMBER OF GROUPS FOR LD₅₀: 2

WEIGHT RANGE (at initiation) Male: 200 -300 grams Female: 200 -300 grams

DIET: Standard laboratory feed for rodents; food and water were available ad libitum.

TEST MATERIAL AND DOSE LEVEL FOR LD₅₀: finely ground white hard core cellulosic material, obtained from Dekalb maize hybrid DK 446, mixed with 1% weight “black strap” molasses, the mixture being mixed with Tween 80® at a 1:2 (w:v) concentration in a dose level of 5 g/kg and 15.1 g/kg for the respective groups.

FREQUENCY AND ROUTE OF ADMINISTRATION: Once every 24 hours for the 5 g/kg dose level; at the 15.1 g/kg dose level each animal was given a portion of its dose at two dosing points within approximately four hours. The doses were administered orally by syringe and suitable incubation tube.

STUDY PERIOD: 14 days

### RESULTS

There were no mortalities in the 5 g/kg dose level group. In the 15.1 g/kg dose level group the animals that died immediately after dosing due to misdosing or anomaly from dosing were replaced. No mortality of surviving animals or replaced animals occurred during the study period.

All surviving animals in all dose groups had a weight gain by day 14, as exemplified in Table I below:

TABLE I
Dose group: 15.1 g/kg

| Rat | Sex | Initial Wt (g) | Final Wt (g) | % change in Wt |
|---|---|---|---|---|
| 1 | F | 234 | 278 | +44 |
| 2 | F | 224 | 264 | +40 |
| 3 | F | 227 | 266 | +39 |
| 4 | F | 224 | 260 | +36 |
| 5 | F | 225 | 266 | +41 |
| 6 | M | 246 | 351 | +105 |
| 7 | M | 250 | 354 | +104 |
| 8 | M | 267 | 382 | +115 |
| 9 | M | 270 | 384 | +114 |
| 10 | M | 230 | 356 | +126 |

There were no clinical abnormalities in the 5.0 g/kg group, clinical observations in the 15.1 g/kg group included rales, lethargy, diarrhoea and anogenital staining.

### CONCLUSION

The material did not produce compound-related mortality in half or more of the animals, even at a dose level of 15.1 g/kg, and can therefore be considered practically non-toxic.

Efficacy Study

TEST ANIMALS: Sprague Dawley derived Rattus norvegicus

NUMBER AND SEX (in each group): 5 Male & 5 Female (females nulliparous and non-pregnant)

NUMBER OF GROUPS: 1

WEIGHT RANGE (at initiation) Male: 115-125 grams Female: 115-125 grams

DIET: Standard laboratory feed for rodents; food and water were available ad libitum.

TEST MATERIAL: Pellets obtained by extrusion of the finely ground white hard core cellulosic material from the hybrid used in the above toxicity study, mixed before extrusion with 1% by weight “black strap” molasses.

FREQUENCY AND ROUTE OF ADMINISTRATION: The test material was placed in 113 g (4 ounce) clear glass feeding jars for continuous ad libitum access to the food. Additional material was added daily and an equal quantity was given to each animal.

STUDY PERIOD: 14 days

### RESULTS

All animals died by day 7. On day 4, one female was found dead. On day 5, two males were found dead and one female was found dead. On day 6, two males and two females were found dead. On day 7, one male and one female were found dead.

Clinical observations included dehydration, lethargy, tremors, weight loss, hunching and soft light stool.

All animals had a daily weight loss, as illustrated in Table II below:

TABLE II
Body weight data (g)

| Rat | Sex | DAY 0 | DAY 3 | DAY 7 |
|---|---|---|---|---|
| 1 | F | 119 | 93 | (found dead on day 6) |
| 2 | F | 120 | 91 | (found dead on day 5) |
| 3 | F | 117 | 84 | (found dead on day 4) |
| 4 | F | 122 | 96 | (found dead on day 7) |
| 5 | F | 119 | 96 | (found dead on day 6) |
| 6 | M | 120 | 99 | (found dead on day 6) |
| 7 | M | 121 | 91 | (found dead on day 5) |
| 8 | M | 123 | 94 | (found dead on day 5) |
| 9 | M | 123 | 101 | (found dead on day 6) |
| 10 | M | 119 | 97 | (found dead on day 7). |

It is clear that when administered in a manner enabling free access, the test material has a powerful rodenticidal effect.

The invention also extends to a rodenticide comprising any material (particularly but not necessarily any material which is non-toxic at a controlled dosage level of up to 15 g/kg per day) which is an agonist in rodents of that cellulosic white core material obtained from the DK 446 hybrid which is rodenticidal when administered in a manner enabling free access to rodents.

The invention extends to rodenticides comprising any of the cellulosic rodenticidal materials identified above, whether synthetic or obtained from natural sources.

The bait attractant may optionally comprise crushed whole wheat and/or crushed oats as an alternative to or in addition to the other bait attractants referred to above.

1-13. (canceled)

14. A rodenticide comprising cellulosic rodenticidal material obtained from hybrid corn cobs which material is non-toxic to humans but which causes rodents to lose weight and die.

15. A rodenticide according to claim 14 wherein the rodenticidal material is obtained from cobs of hybrid maize, said hybrid maize normally growing to a height of 2.7 to 3.3 metres.

16. A rodenticide according to claim 14 wherein the rodenticidal material is obtained from cobs of hybrid maize, said hybrid maize normally having a single giant ear of corn.

17. A rodenticide according to claim 14 which also includes a sweet material which acts as a bait attractant.

18. A rodenticide according to claim 17 wherein said sweet material is selected from the group consisting of ground sugar beet and unrefined molasses.

19. A rodenticide according to claim 14 wherein the material is non-toxic at a controlled dosage level of up to 15 g/kg per day.

20. The rodenticide of claim 14 wherein the cellulosic material consists essentially of ÿ cellulose.

21. A method of alleviating rodent infestation, the method comprising depositing in a region of infestation a rodenticide comprising cellulosic material obtained from hybrid corn cobs.

22. A method for killing rodents which comprises administering thereto an effective amount of a rodenticide according to claim 14.

23. The method according to claim 21 wherein said cellulosic material consists essentially of cellulose.

24. A method according to claim 21 wherein a sweet material is also utilized as a bait attractant.

25. A method of making a rodenticide which comprises mixing together a bait attractant and cellulosic material obtained from hybrid corn cobs.

26. The method as claimed in claim 25 wherein said cellulosic material is obtained from the cob of a corn hybrid characterized by normally growing to a height of 2.7 to 3.3 metres (9 to 11 feet) and by normally having a single giant ear of corn.

27. A method for killing rodents which comprises orally administering to the rodents an effective amount of a composition consisting essentially of ÿ cellulose particles derived from hybrid corn cobs and mixed with an attractant, without the addition of pesticide or poison.

28. The method as defined in claim 27 in which the cellulose particles are bound together with the attractant.

29. The method as defined in claim 27 in which the cellulose comprises about 99% of the composition.

30. The method as defined in claim 27 in which the cellulose is crushed to a size that can be ingested by the rodent.
